(19) United States
(12) Patent Application Publication
(10) Pub. No.: US 2025/0064675 A1
(43) Pub. Date: Feb. 27, 2025

(54) HAND WEARABLE APPARATUS, BRACELET AND WEARABLE APPARATUS FOR AREA BETWEEN THUMB AND FOREFINGER

(71) Applicant: Ningbo Kangluo Intelligent Technology Co.,Ltd., Ningbo (CN)

(72) Inventor: Hao Zheng, Ningbo City (CN)

(21) Appl. No.: 18/543,080

(22) Filed: Dec. 18, 2023

Related U.S. Application Data
(63) Continuation-in-part of application No. 18/236,918, filed on Aug. 22, 2023, now Pat. No. 12,029,860.

(51) Int. Cl. A61H 39/00; A61H 39/04

(52) U.S. Cl. CPC A61H 39/002; A61H 39/04; A61H 2201/0103; A61H 2201/1207; A61H 2201/1635; A61H 2201/165; A61H 2201/5007; A61H 2201/5043; A61H 2201/5082

(57) ABSTRACT

The present disclosure provides a hand wearable apparatus, a bracelet, and a wearable apparatus for an area between a thumb and a forefinger. The hand wearable apparatus includes a wearable part configured to be worn on a hand; an electrode assembly disposed on the wearable part and configured to abut a skin surface of a human body and provide a micro current to the skin surface; and at least one driving device disposed on the wearable part configured to drive the electrode assembly to press the skin surface.

### CROSS-REFERENCE TO RELATED APPLICATIONS

The application is a Continuation-in-Part of the U.S. application Ser. No. 18/236,918 filed on 22 Aug. 2023, and entitled “A HAND WEARABLE APPARATUS, A BRACELET AND A WEARABLE APPARATUS FOR A HEGU POINT AREA” now pending, the entire disclosures of which are incorporated herein by reference.

### TECHNICAL FIELD

The present disclosure relates to the technical field of wearable products, and in particular, to a hand wearable apparatus, a bracelet, and a wearable apparatus for an area between a thumb and a forefinger.

### BACKGROUND

At present, an existing electrical stimulation used on acupuncture points is generally used to provide a micro current to the electrode assembly in order to cooperate with the human body, which is a conductive, to achieve the effect of physical therapy. The existing electrode assembly is generally attached to a surface of human skin in a form of a fastening band which will press the skin surface in large area and easily bring discomfort to a user. However, in this way, the electrode assembly cannot fit tightly on the skin surface, so it will weaken the effect of the electrical stimulation.

### SUMMARY

A main objective of the present disclosure is to provide a hand wearable apparatus, a bracelet, and a wearable apparatus for an area between a thumb and a forefinger, so as to resolve a problem that a fixing manner of an electrode assembly in an existing electrical stimulation is easy to bring discomfort to a user and does not fit a skin surface well to affect a use effect.

To resolve the foregoing technical problem, technical solutions are provided in the present disclosure.

According to one aspect, the present disclosure provides a hand wearable apparatus, which includes a wearable part configured to be worn on a hand; an electrode assembly disposed on the wearable part, wherein the electrode assembly is configured to abut a skin surface of a human body and provide a micro current to the skin surface; and at least one driving device disposed on the wearable part, wherein the driving device is configured to drive the electrode assembly to press the skin surface of the human body.

In one embodiment, the driving device includes a first airbag and an air pump configured to inflate or deflate the first airbag, and the first airbag is configured to drive the electrode assembly to press the skin surface during inflating or deflating.

In one embodiment, the driving device includes an air pump and a cylinder connected to the air pump by using an air pipe, and the cylinder is configured to drive the electrode assembly to press the skin surface when the air pump blows air to the cylinder.

In one embodiment, the driving device includes a motor, a screw connected to an output shaft of the motor, a screw nut cooperated with the screw, and a slider disposed on the screw nut, and the slider is configured to drive the electrode assembly to press the skin surface when the screw rotates.

In one embodiment, the driving device includes a motor, a gear connected to the motor, and a rack engaged with the gear; and the rack is configured to drive the electrode assembly to press the skin surface when the gear rotates.

In one embodiment, the driving device includes an electric push rod, and when the electric push rod works, the electrode assembly is driven to press the skin surface.

In one embodiment, the driving device includes a coil and a magnet, and when the coil is powered on, the magnet moves to drive the electrode assembly to press the skin surface.

In one embodiment, the driving device includes a motor and a crank electrically connected to the motor, and the crank is configured to drive the electrode assembly press to the skin surface when the motor is driven.

In one embodiment, the driving device includes a motor, a gear connected to the motor and having a screw, a screw nut movably disposed on the screw, and a slider disposed on the screw nut; and the gear is configured to drive the screw to rotate when the motor is driven, and the screw nut is configured to drive the slider to move when the screw is rotated, so as to drive the electrode assembly to press the skin surface.

In one embodiment, the driving device includes a motor, a worm connected to the motor, and a screw rod connected to the worm; and the screw rod drives the electrode assembly to press the skin surface when the worm rotates.

According to still another aspect, the present disclosure further provides a bracelet, including: a wrist strap; a dial connected to the wrist strap; an electrode assembly, wherein the electrode assembly is configured to press a skin surface of a human body and provide a micro current to the skin surface; at least one driving device, wherein the driving device is configured to drive the electrode assembly to press the skin surface; and a controller electrically connected to the driving device and the electrode assembly respectively.

In one embodiment, the electrode assembly is disposed on a driving device, and the driving device is disposed on the wrist strap or the dial.

In one embodiment, the electrode assembly is disposed opposite to a driving device, and the driving device is disposed on the wrist strap or the dial.

In one embodiment, the driving device includes at least one first airbag and at least one air pump electrically connected to the controller, and the first airbag is configured to drive the electrode assembly to press the skin surface of when the air pump is driven.

In one embodiment, the first airbag and the electrode assembly are disposed in the wrist strap or the dial.

In one embodiment, the first airbag is disposed in a dial, and the electrode assembly is disposed on the wrist strap.

In one embodiment, the first airbag is disposed on the wrist strap, and the electrode assembly is disposed on the dial.

In one embodiment, each first airbag is disposed in both the wrist strap and the dial, and the electrode assembly is disposed on the wrist strap or the dial.

In one embodiment, the bracelet further includes a second airbag configured to press an acupuncture point or a protrusion element for pressing an acupuncture point.

In one embodiment, the second airbag is an inflatable airbag.

In one embodiment, the first airbag and the second airbag are in an integral structure, the first airbag communicates the second airbag, and an inflation and deflation opening is disposed on the first airbag and/or the second airbag.

In one embodiment, further including a pressure sensor and an air pressure sensor, wherein the pressure sensor and the air pressure sensor are electrically connected to the controller; the pressure sensor is configured to obtain a contact pressure value between the electrode assembly and the skin surface and transmit the contact pressure value to the controller, the controller is configured to generate a driving data for controlling the air pump based on the contact pressure value; the air pump is configured for inflating or deflating the first airbag according to the driving data, so that the electrode assembly is press the skin surface; the air pressure sensor is configured to generate a barometric pressure value by detecting the driving device and transmit the barometric pressure value to the controller; and the controller is also configured to determine the driving data for the driving device and an electrical stimulation signal for implementing electrical stimulation providing to the electrode assembly according to the atmospheric pressure value and the contact pressure value.

In one embodiment, the controller is further configured to determine the contact pressure value between the electrode assembly and the skin surface of the human body is less than or greater than a preset pressure value, and generate a reminder signal for reminding the user to adjust the bracelet when the contact pressure value between the electrode assembly and the skin surface is less than or greater than the preset pressure value.

In one embodiment, further including a display screen electrically connected to the controller, wherein the display screen is configured to display a display result of a target task in response to a display instruction of a user for the target task, and the display result includes at least one or more of the following information: an intensity of the electrical stimulation, a squeezing strength of the driving device, an countdown protection information, a breathing mode, or user health data.

In one embodiment, the display screen is a touch display screen; and the touch display screen is configured to display a control result of the target task in response to a touch control instruction of the user for the target task; and the control result includes one or more of changing the intensity of the electrical stimulation, a squeezing strength of the driving device, a countdown protection information, and time under the touch control instruction.

In one embodiment, a pressure sensor electrically connected to the controller is disposed in the first airbag; the pressure sensor is configured to detect a pressure value inside the first airbag and transmit the pressure value to the controller, and the controller determines air pressure controlling data for controlling the air pump according to the pressure value and the contact pressure value.

In one embodiment, further including a temperature sensor, a heating component, and a refrigeration component electrically connected to the controller: wherein the temperature sensor is configured to detect a temperature value between the electrode assembly and the skin surface, and transmit the temperature value to the controller; the controller is configured to determine a temperature adjustment policy for the first airbag according to the temperature value, and/or an electrical stimulation policy for the electrode assembly; and the controller is further configured to control the heating component to heat air in the first airbag; and the refrigeration component is configured to cool the bracelet when an electrical stimulation is applied to the skin surface by the electrode assembly.

In one embodiment, further including a breathing adjustment component electrically connected to the controller; and the breathing adjustment component configured to remind the user to adjust respiration according to a preset breathing mode when an electrical stimulation is applied to the skin surface by the electrode assembly.

In one embodiment, further including a health data collection component electrically connected to the controller; and the health data collection component configured to collect, in real time or periodically, health data of the user, wherein the health data includes at least one or more of the following data: a temperature value, a blood pressure value, a blood oxygen value, and a heartbeat value; and the controller provides a health analysis result and/or a health report based on the health data.

In one embodiment, the controller is also configured to acquire driving data for the driving device; the controller is also configured to apply an electrical stimulation signal to the skin surface via the electrode assembly when the driving device drives the electrode assembly to press the skin surface; and the controller is also configured to apply the electrical stimulation signal to the electrode assembly when a preset electrical stimulation condition is reached.

In one embodiment, further including a switch component electrically connected to the controller; and the switch component configured to acquire a switch signal triggered by the user and transmit the switch signal to the controller; the controller configured to determine a switch state for the bracelet based on the switch signal, and/or determine an electrical stimulation mode and an intensity of an electrical stimulation of the electrode assembly.

In one embodiment, further including a rotary encoder electrically connected to the controller; and the rotary encoder is configured to: based on a rotation adjustment operation, determine an intensity of an electrical stimulation for the electrode assembly based on a change amount indicated by the rotation adjustment operation, so that the controller applies an electrical stimulation signal matching the intensity of the electrical stimulation to the electrode assembly; or determine a squeezing strength of the driving device, so that a matching pressing force to the electrode assembly is controlled; or determine a time or an countdown protection information, so that a matching pressing time of the electrode assembly is controlled.

According to another aspect, the present disclosure further provides a wearable apparatus for an area between a thumb and a forefinger including: a wearing belt used for wearing; an electrode assembly disposed on the wearing belt, wherein the electrode assembly is configured to abut a skin surface of a human body and provide a micro current to the skin surface, and the electrode assembly is located at a position correspond to an area between the thumb and the forefinger when the wearing belt is worn on a hand of a user; and at least one driving device disposed on the wearing belt, wherein the driving device is configured to drive the electrode assembly to press the skin surface.

The present disclosure has the following beneficial effects: compared with the prior art, in the present disclosure, the driving device is configured to drive the electrode assembly to abut to the skin surface of the human body, so as to reduce an existing large area of pressing on the skin surface, and improve a feeling of comfort when a user uses the hand wearable apparatus, so that a stable contact can be maintained between the skin surface and the electrode assembly, thereby ensuring effectiveness of an electrical stimulation, and significantly improving an electrical stimulation effect.

### BRIEF DESCRIPTION OF THE DRAWINGS

In order to explain the technical solutions of the embodiments of the present disclosure more clearly, the following will briefly introduce the accompanying drawings used in the embodiments. Apparently, the drawings in the following description are only some embodiments of the present disclosure. Those of ordinary skill in the art can obtain other drawings based on these drawings without creative work.

FIG. 1 is a module diagram of a hand wearable apparatus of the present disclosure.

FIG. 2A to FIG. 2D are structural diagrams of a hand wearable apparatus according to a first embodiment of the present disclosure.

FIG. 3A to FIG. 3E are schematic diagrams of the specific setting position of the first airbag and electrode assembly of the embodiment of the present disclosure.

FIG. 4A to FIG. 4B are structural diagrams of a hand wearable apparatus according to a second embodiment of the present disclosure.

FIG. 5A to FIG. 5C are structural diagram of a hand wearable apparatus according to a third embodiment of the present disclosure.

FIG. 6A to FIG. 6D are structural diagrams of a hand wearable apparatus according to a forth embodiment of the present disclosure.

FIG. 7A to FIG. 7B are structural diagrams of a hand wearable apparatus according to a sixth embodiment of the present disclosure.

FIG. 8A to FIG. 8C are structural diagrams of a hand wearable apparatus according to a seventh embodiment of the present disclosure.

FIG. 9A to FIG. 9D are structural diagrams of a hand wearable apparatus according to a eight embodiment of the present disclosure.

FIG. 10A to FIG. 10D are structural diagrams of a hand wearable apparatus according to a ninth embodiment of the present disclosure.

FIG. 11 is a diagram of a curve of an air pressure variation in a first air bag according to the second embodiment of the present disclosure.

FIG. 12A to FIG. 12D are diagrams of a bracelet according to an embodiment of the present disclosure.

FIG. 13 is a diagram of a wearable apparatus for an area between a thumb and a forefinger according to an embodiment of the present disclosure.

### DETAILED DESCRIPTION OF THE EMBODIMENTS

The accompanying drawings in the embodiment of the present disclosure are combined, The technical scheme in the embodiment of the present disclosure is clearly and completely described, Obviously, the described embodiment is only a part of the embodiment of the present disclosure, but not all embodiments are based on the embodiment of the present disclosure, and all other embodiments obtained by ordinary technicians in the field on the premise of not doing creative work belong to the protection range of the present disclosure.

Currently, an electrode plate on the market and used to stimulate a physical therapy action is generally applied to a skin surface of a human body in a form of a fastening band. However, overall discomfort is easily imposed on a user, and is not applicable to wear for a long time, and the electrode plate is not necessarily able to properly fasten the skin surface of the human body or the like.

Therefore, this embodiment provides a hand wearable apparatus.

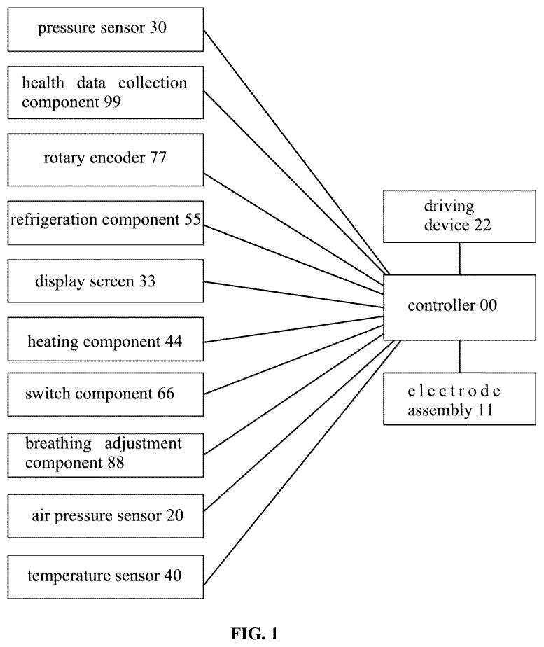

Referring to FIG. 1, the hand wearable apparatus includes an electrode assembly 11 and several driving devices 22. The electrode assembly 11 is configured to abut a skin surface of a human body and provide a micro current to the skin surface, so as to stimulate a human nerve by using the micro current, so as to implement a physiotherapy function. The driving device 22 is configured to drive the electrode assembly 11 to press the skin surface of the human body, so as to reduce an existing large area of pressing on the skin surface, improve a feeling of comfort when a user uses the hand wearable apparatus, so that a stable contact can be maintained between the skin surface and the electrode assembly 11, thereby ensuring effectiveness of an electrical stimulation, and significantly improving an electrical stimulation effect.

In practical application, the hand wearable apparatus can be applied to finger wearable devices, specifically gloves. Therefore, when the electrode assembly 11 is set on the corresponding finger or palm acupuncture points, it can be pressed on the corresponding acupuncture point in conjunction with the driving device 22. When the electrode assembly 11 is placed on the glove and corresponding to a Shangyang acupuncture point on the index finger, constipation can be regulated by a pressing and electrical stimulation of the electrode component 11. When the electrode assembly 11 is placed on a glove and corresponds to a Hegu point area, the pressing and electrical stimulation of the electrode assembly 11 can help to relax the muscles, activate the meridians, and enhance the body's resistance, achieving therapeutic effects.

Specifically, the hand wearable apparatus may be applied to a bracelet, that is, the bracelet includes the hand wearable apparatus described above. The wearable member 10 includes a wrist strap 7 configured to be disposed on a wrist of the human body. When the user wears the wrist strap on the wrist, the electrode assembly 11 may be aligned with an acupuncture point on the wrist of the human body, so that when the driving device 22 is driven, the electrode assembly 11 is brought into abutment with the skin surface corresponding to the acupuncture point, for example, Neiguan acupuncture point. Therefore, by the micro current, an antiemetic effect (such as a halo prevention effect) may be implemented, and it is also easy to carry. The electrode assembly 11 may be one or more electrode plates, and a size of the electrode plate may be defined by a manufacturer.

Specifically, the electrode assembly 11 is disposed on the driving device 22, that is, the electrode assembly 11 is indirectly disposed on the wrist strap 7. When the driving device 22 drives, the electrode assembly 11 may be directly driven to move against the skin surface. In this embodiment, both the driving device 22 and the electrode assembly 11 may be disposed on the wrist strap 7 or the dial 1. In another embodiment, the driving device 22 is disposed opposite to the electrode assembly 11. That is, the driving device 22 is disposed on the wrist strap 7, and the electrode assembly 11 may be disposed on the dial 1. Alternatively, the electrode assembly 11 is disposed on the wrist strap 7, and the driving device 22 may be disposed on the dial 1. When the driving device 22 is driven, the wrist strap 7 at an opposite side surface may be tightened by using a reaction force, or the electrode assembly 11 may press against the skin surface.

To implement control over the electrode assembly 11 and the driving device 22, the bracelet in this embodiment further includes a controller 00, and the controller 00 is electrically connected to the electrode assembly 11 and the driving device 22 respectively. The controller 00 is configured to: generate a driving data for the driving device 22 and transmit the driving data to the driving device 22. The driving device 22 is configured to make the electrode assembly 11 close to the skin surface of the human body according to the driving data. The electrode assembly 11 is configured to apply an electrical stimulation to the skin surface based on the current signal output by the controller 00 when the skin surface is pressed tightly the skin surface by the the electrode assembly 11.

To facilitate understanding of the bracelet provided in this embodiment of the present disclosure, the following briefly describes an application scenario of the bracelet. The bracelet may be mainly applied to a related scenario in which a vehicle, a ship, an airplane, or the like moves. This is mainly because in these related scenarios, discomfort of the human body, such as a vertigo feeling or a vomiting feeling, can be stimulated by acceleration movement in any form, such as bumpy, swaying, or rotating, so as to relieve discomfort of the human body in the foregoing application scenario. Only in this case, the bracelet is provided.

It should be noted that the bracelet herein may be further applied to another scenario, for example, for a pregnant woman in a puerperal period, the bracelet provided in this embodiment of the present disclosure may be effectively used to stop emesis. In addition, the bracelet may be further applied to another related scenario, for example, used for pressure relief or hand trembling relief used for ET trembling, which is not specifically limited herein. In consideration of a general situation of vehicles such as seasickness and seasickness, these scenarios are also used as examples for description later.

To avoid, as far as possible, a problem of relatively poor stimulation effectiveness caused by improper bonding between the bracelet and the skin surface of the human body, in this embodiment of the present disclosure, an effect of pressing the skin surface of the human body by the bracelet may be improve by a cooperation work between the controller 00, the driving device 22, and the electrode assembly 11, an electrical stimulation in an pressing state is implemented.

In some embodiments, the driving device 22 may be driven, so that the electrode assembly 11 in a driving state can better adhere the skin surface of the human body, and a contact force is increased. In this case, the electrical stimulation is performed to better alleviate symptoms such as sickness, and a sickness prevention effect is stronger.

The electrode assembly 11 in this embodiment of the present disclosure may include, for example, a single electrode plate, or may be multiple electrode plates (for example, an electrode group formed by two electrode plates). The driving device 22 herein may match one or more driving elements, which is not specifically limited herein.

In some embodiments, the controller 00 may determine the driving data for the driver based on a contact pressure value between the electrode plate and the skin surface of the human body, and may cause, according to the driving data, a bracelet to produce an effect of pressing the skin surface of the human body tightly.

The driving data herein is mainly related a control data that causes a change of the driving device 22. For example, in a case in which the driving device 22 includes the first airbag 221b, the control data herein may be an inflatable amount of the first airbag 221b, so as to control a contact strength between the first airbag 221b and the skin surface, thereby controlling contact the strength between the electrode assembly 11 and the skin surface of the human body. The number of first airbags 221b is multiple, and the number of electrode pieces of the electrode assembly 11 is equal to the number of first airbags 221b.

In a case in which it is determined that a change generated by the driving device 22 causes enough contact pressure, an electrical stimulation may be applied to the electrode assembly 11 by the controls of the controller 00.

Specifically, the electrode assembly 11 may be disposed on the first airbag 221b, or on a side that is disposed on the wrist strap 7 and opposite to the first airbag 221b.

In any one of the foregoing cooperation manners, compared with a solution that is fastened only by a wrist strap, the bracelet provided in the present disclosure does not need to be fastened and worn, so that loose wearing of the bracelet can still ensure that the electrode assembly 11 press the skin surface tightly and a stable contact between the bracelet and the electrode assembly 11 is maintained, and a comfortable feeling can be ensured when an effectiveness electrical stimulation is provided.

In addition, in a pressing state, the electrode assembly 11 ejects into a depressed portion o the skin surface, so that the electrode assembly 11 is closer to a median nerve in a deep part of the wrist of the human body, an electric field of the electrical stimulation propagates more efficiently in the organism, and a better stimulation effect is provided by using the electric stimulation with a power same as the prior art, thereby further improving an stimulation effectiveness of the bracelet.

Considering that the driving device 22 plays a key role in the bracelet provided in this embodiment of the present disclosure, the following focuses on the driving device 22.

The driving device 22 herein may be specifically implemented by using multiple structures. This embodiment of the present disclosure mainly provides the following nine implementation manners.

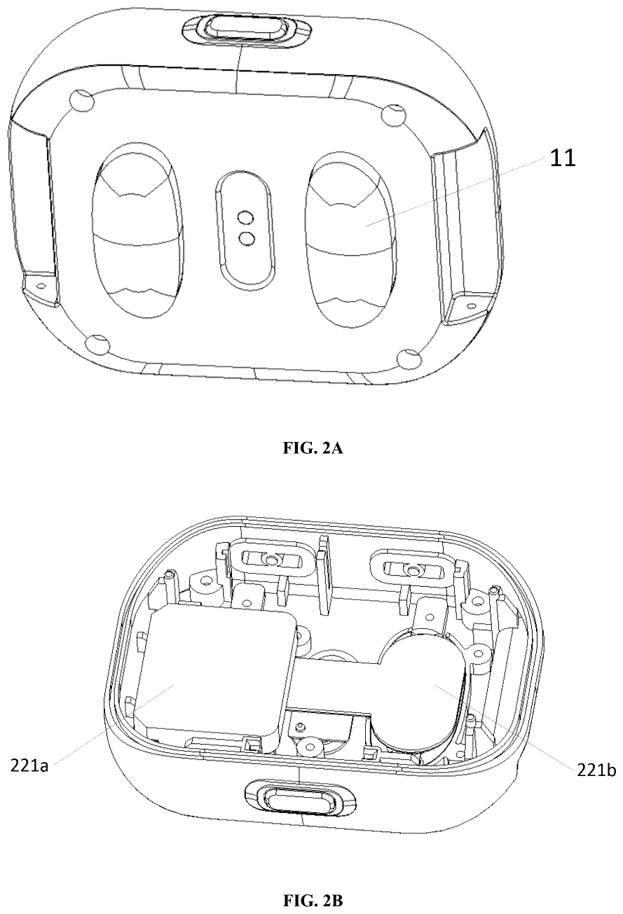

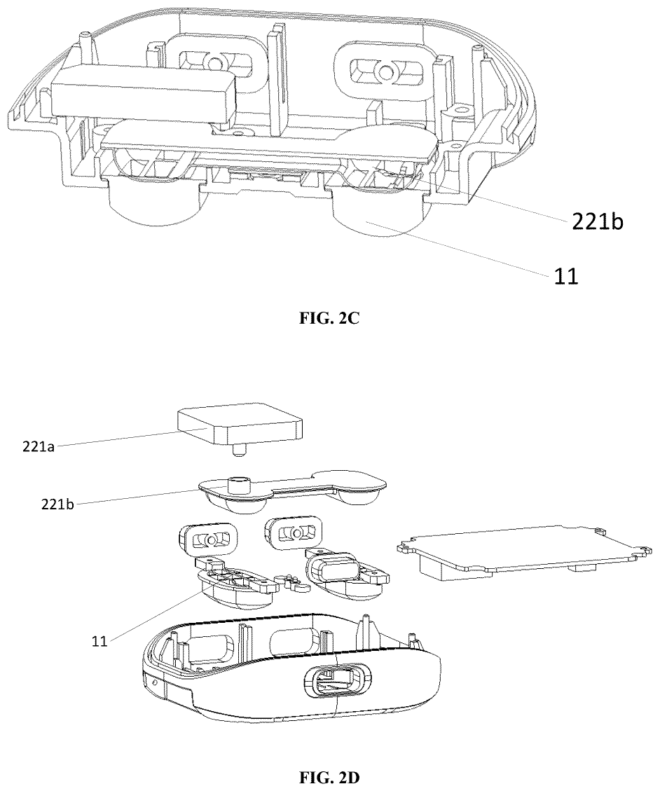

Manner 1: referring to FIG. 2A to FIG. 2D, the driving device 22 includes an air pump 221 an and a first airbag 221b.

The air pump 221a is electrically connected to the controller 00. The air pump 221a inflates the first airbag 221b according to the driving data. After the first airbag 221b is deformed, the electrode assembly 11 is driven to approach and squeeze the skin surface of the human body. Alternatively, the first airbag 221b directly presses the skin surface of the human body after being deformed, so that the electrode assembly 11 located on the opposite side is close to the skin surface of the human body by using a reaction force.

To determine a more accurate deformation amount of the first airbag 221b, a pressure sensor may be used herein to obtain a contact pressure value between the electrode assembly 11 and a skin surface of the human body in real time or non-real time (for example, timing). A greater pressure value indicates that the electrode assembly 11 is relatively tight to the skin surface of the human body. In this case, a relatively small deformation amount is required, and a smaller pressure value indicates that the electrode assembly 11 is relatively loose to the skin surface of the human body. In this case, a relatively large deformation amount is required.

As shown in FIG. 2A to FIG. 2C, both the first airbag 221b and the electrode assembly 11 are disposed in the controller 00. In addition, the first airbag 221b and the electrode assembly 11 may be disposed according to different application requirements. For example, the first airbag 221b is disposed on the wrist strap 7 and is different from the controller 00, and the electrode assembly 11 is disposed on the first airbag 221b. For example, the first airbag 221b is disposed in the controller 00, and the electrode assembly 11 is disposed in a position in which the wrist strap 7 is different from the controller 00. For example, the first airbag 221b is disposed on the wrist strap 7 but not on the controller 00, and the electrode assembly 11 is disposed on the controller 00. For example, the first airbag 221b is disposed on both the wrist strap 7 and the controller 00, and the electrode assembly 11 is disposed on the wrist strap 7, the controller 00, or the first airbag 221b.

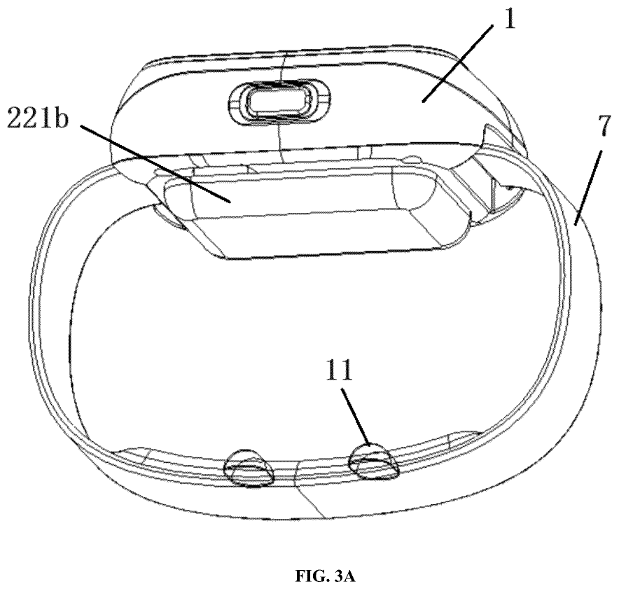

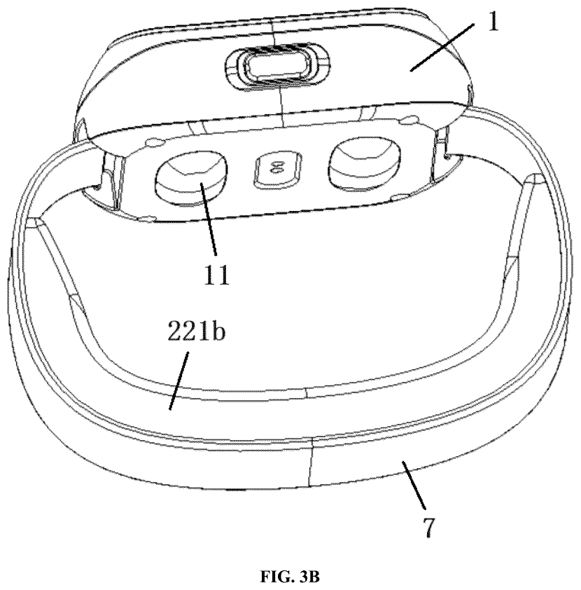

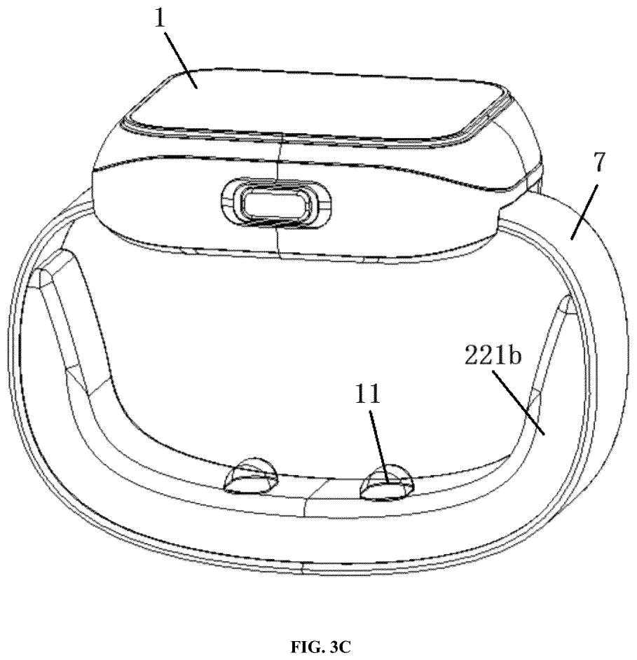

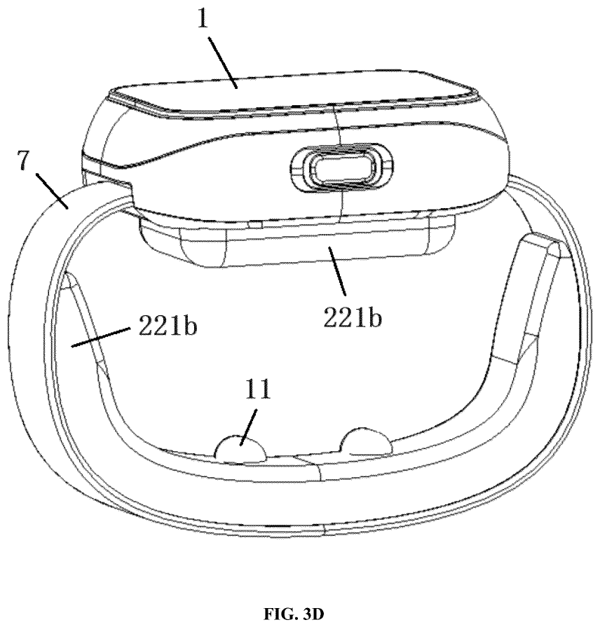

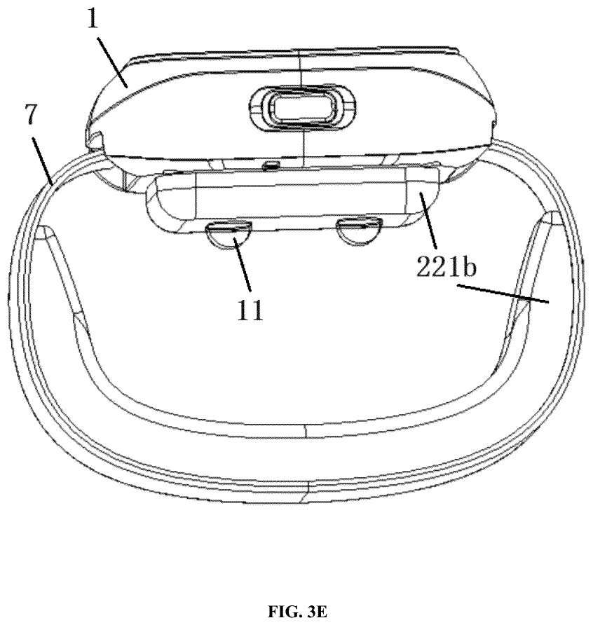

As shown in FIG. 3A to FIG. 3E, the quantity and location of the first air bag 221b and the electrode assembly 11 in the bracelet provided in this embodiment of the present disclosure may be set as required, which is not specifically limited.

As shown in FIG. 2D, FIG. 2D is an expanded structural diagram of the driving device 22 of the foregoing first manner according to an example of an embodiment of the present disclosure. The structural diagram is only in an example form. In this application, the structural diagram may be in another form, which is not specifically limited herein.

In a second manner, the driving device 22 includes an air pump 222 an and a cylinder 222b connected to the air pump 222a by using the air pipe. The cylinder 222b drives the electrode assembly 11 to abut the skin surface when the air pump 222a blows the air to the cylinder 222b.

The air pump 222a is electrically connected to the controller 00. Specifically, the air pump 222a determines a displacement of an internal piston of the cylinder 222b according to the driving data. The cylinder 222b drives the internal piston to move according to a moving amount, and directly pushes the electrode assembly 11 toward an inside of the bracelet to produce an effect of pressing the skin surface of the human body tightly. Alternatively, the cylinder 222b drives the internal piston to move according to the moving amount to directly or indirectly squeeze the skin surface of the human body, so that moving the electrode assembly 11 toward the inside of the bracelet may also produce an effect of squeezing the skin surface of the human body.

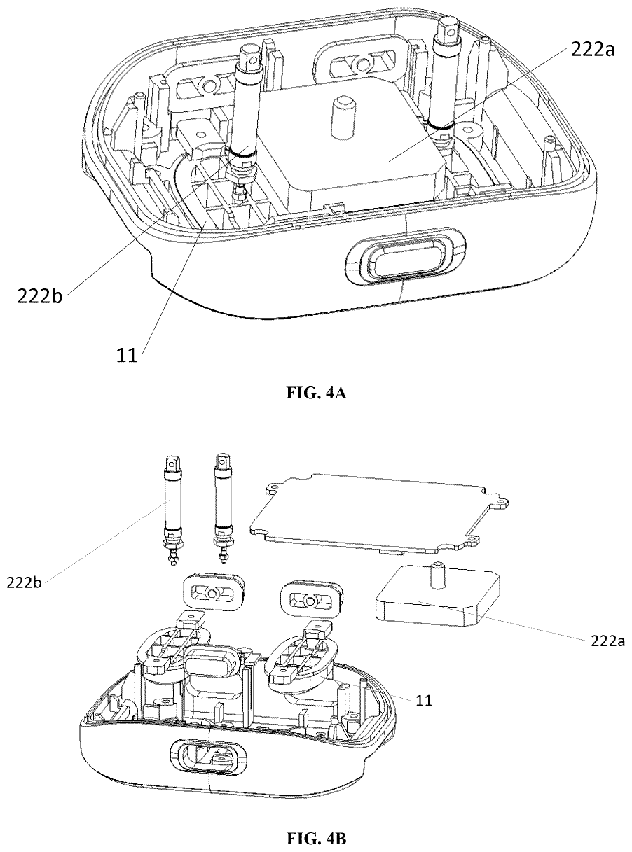

The cylinder 222b herein may use a miniature cylinder. When the cylinder 222a blows air to the miniature cylinder, the piston of the miniature cylinder generates a movement, so as to drive the electrode assembly 11 to move (as shown in FIG. 4A) toward the inside of the bracelet to produce an effect of pressing the skin surface of the human body tightly.

As shown in FIG. 4B, FIG. 4B is an expanded structural diagram of the driving device 22 of the second manner according to an example of an embodiment of the present disclosure. The structural diagram is only in an example form. In actual application, the structural diagram may be in another form, which is not specifically limited herein.

In a third manner, the driving device 22 includes a motor 223a, a screw 223b connected to an output shaft of the motor 223a, a screw nut 223c used to cooperate with the screw 223b, and a slider 223d disposed on the screw nut 223c. When the screw 223b rotates, the slider 223d drives the electrode assembly 11 to abut the skin surface.

The motor 223a is electrically connected to the controller 00, and the motor 223a determines a rotation amount for the screw 223b according to the driving data. The screw 223b drives the screw nut 223c to move according to a rotation amount, so as to move the electrode assembly 11 toward the inner side of the bracelet by using the slider 223d to produce an effect of pressing the skin surface of the human body.

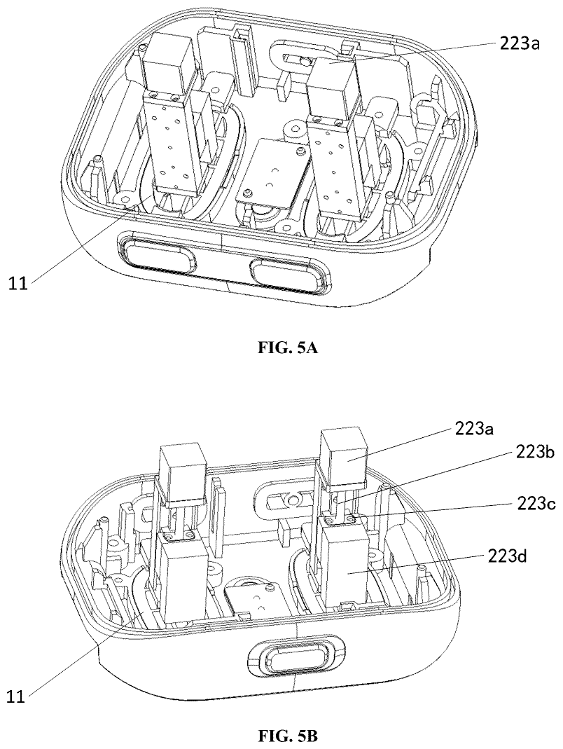

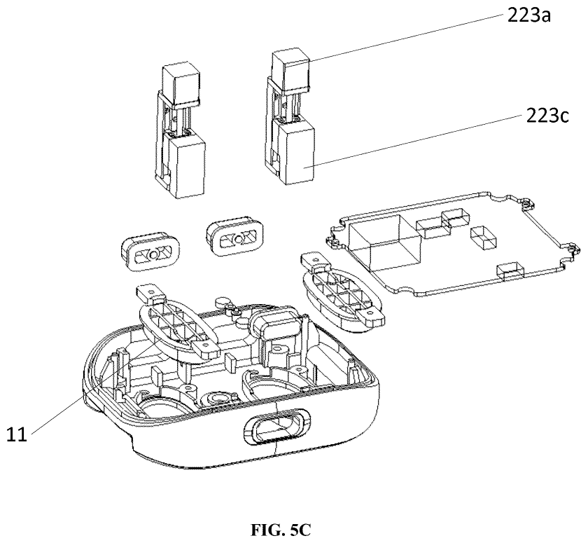

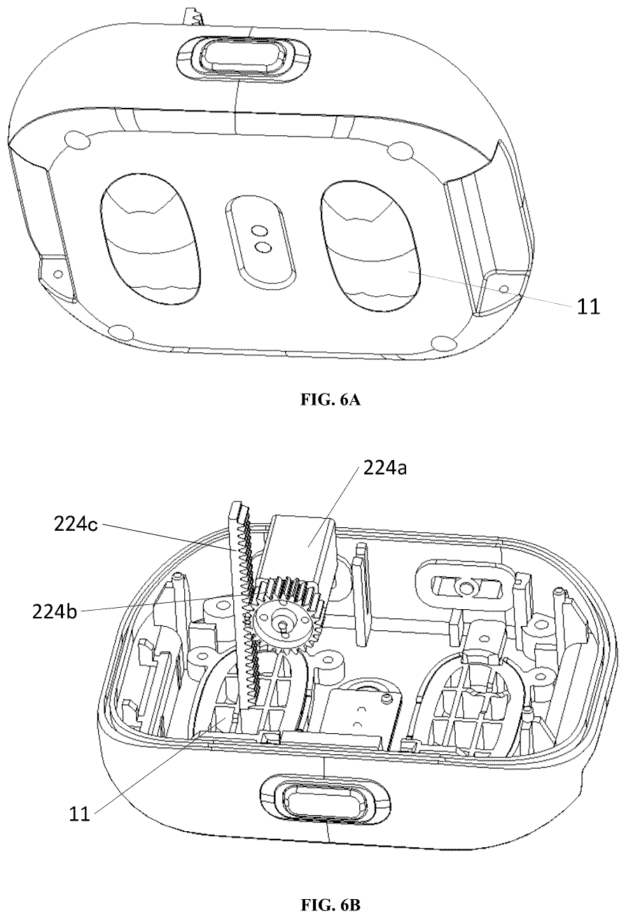

As shown in FIG. 5A and FIG. 5B, the motor 223a drives the screw 223b to rotate, the screw nut 223c on the screw 223b is moved, and the electrode assembly 11 is moved by using the slider 223d to generate an extrusion effect.

As shown in FIG. 5C, FIG. 5C is an expanded structural diagram of the driving device 22 of the foregoing third manner according to an example of an embodiment of the present disclosure. The structural diagram is only in an example form. In actual application, the structural diagram may be in another form, which is not specifically limited herein.

In a fourth manner, the driving device 22 includes a motor 224a, a gear 224b connected to the motor 224a, and a rack 224c engaged with the gear 224b. When the gear 224b rotates, the rack 224c drives the electrode assembly 11 to abut the skin surface, and the motor 224a is electrically connected to the controller 00.

The motor 224a determines a rotation amount for the gear 224b according to the driving data. The gear 224b drives the rack 224c to move according to a rotation amount, so as to drive the electrode assembly 11 to move toward the inner side of the bracelet to produce an effect of pressing the skin surface of the human body.

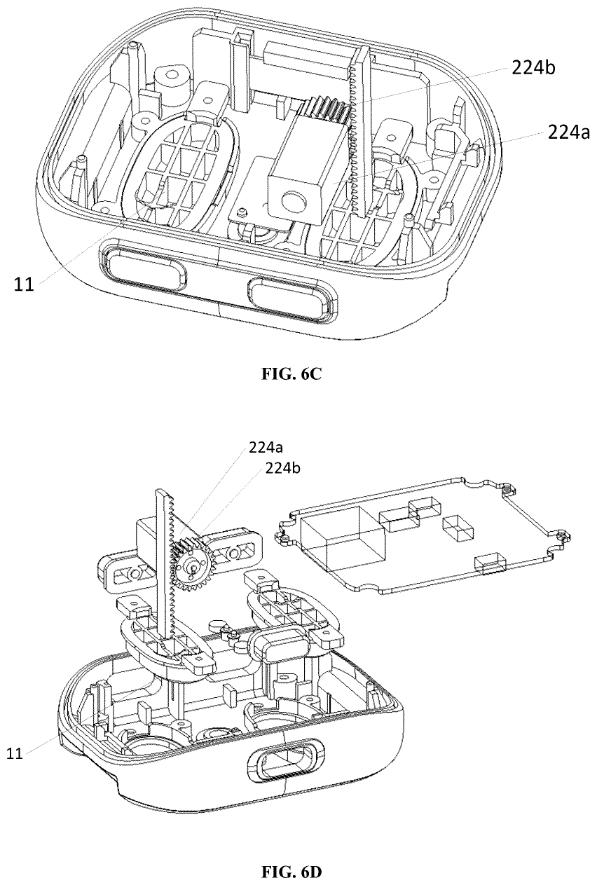

As shown in FIG. 6B and FIG. 6C, the motor 224a drives the gear 224b to rotate, the gear 224b drives the rack 224c to move, and the rack 224c further drives the electrode assembly 11 to move to generate an extrusion effect.

As shown in FIG. 6D, FIG. 6D is an expanded structural diagram of the driving device 22 of the foregoing fourth manner according to an example of an embodiment of the present disclosure. The structural diagram is only in an example form. In actual application, the structural diagram may be in another form, which is not specifically limited herein.

Manner 5: The driving device 22 is an electric push rod, and when the electric push rod works, the electrode assembly 11 is brought into abutment with the skin surface.

The electric push rod is electrically connected to the controller 00. The electric push rod determines a moving amount of an internal piston according to the driving data, so as to drive the electrode assembly 11 to move toward the inner side of the bracelet to produce an effect of pressing the skin surface of the human body tightly. In addition, the electric push rod may directly squeeze the skin surface of the human body according to the moving amount, so that the electrode assembly 11 moves toward the inner side of the bracelet to produce an effect of squeezing the skin surface of the human body. Specifically, the electric push rod may use a micro electric push rod.

In actual application, this embodiment of the present disclosure may further use a worm gear reduction electric push rod mode, or may use a motor gear reduction electric push rod mode, or may use another electric push rod mode, which is not specifically limited herein.

In a sixth manner, the driving device 22 includes a coil 226a and a magnet. When the coil 226a is powered on, the magnet moves to drive the electrode assembly 11 to press the skin surface tightly, and the coil 226a is electrically connected to the controller 00.

In a case in which magnetic current is generated by energizing the coil 226a, the electrode assembly 11 moves toward the inside of the bracelet based on the repulsion motion between the coil 226a and the magnet, so as to produce an effect of pressing the skin surface of the human body.

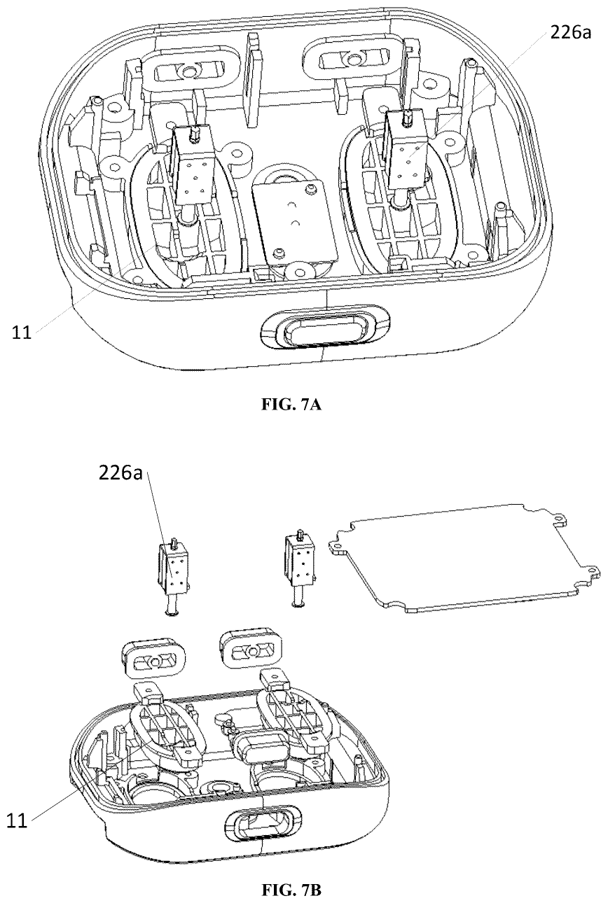

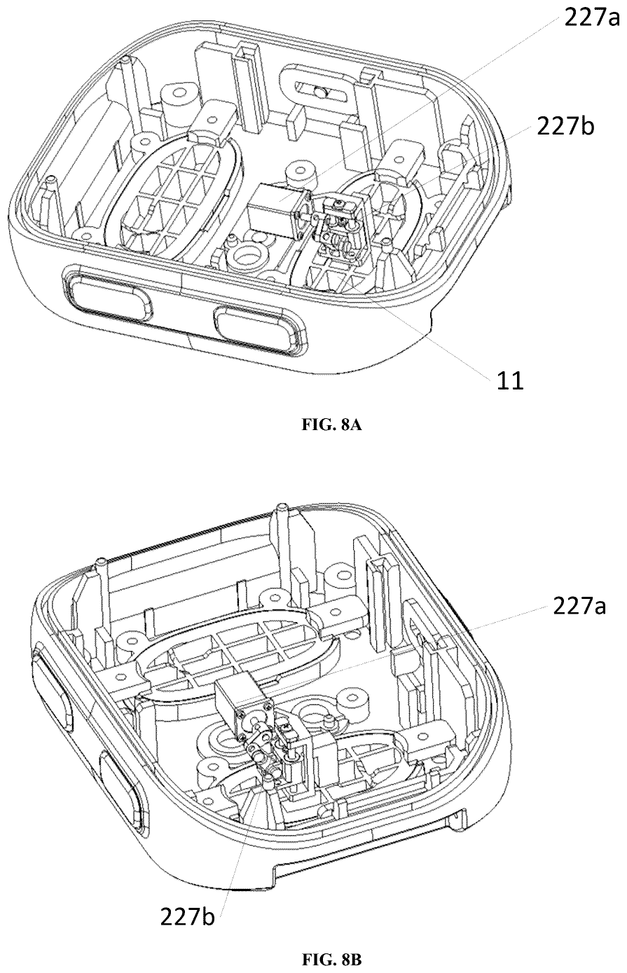

As shown in FIG. 7A, the coil 226a is energized to produce a magnetic field, so that the magnets with the same magnetic field are repulsed and moved so that the electrode assembly 11 generates an extrusion effect.

As shown in FIG. 7B, FIG. 7B is an expanded structural diagram of the driving device 22 of the foregoing sixth manner according to an example of an embodiment of the present disclosure. The structural diagram is only in an example form. In actual application, the structural diagram may be in another form, which is not specifically limited herein.

In another embodiment, the electrode assembly 11 is a magnet, and has a polarity exclusive to the coil 226a.

In a seventh manner, the driving device 22 includes a motor 227 an and a crank 227b that is electrically connected to the motor 227a. The crank 227b drives the electrode assembly 11 to press the skin surface when the motor 227a is driven, and the motor 227a is electrically connected to the controller 00.

The motor 227a determines a rotation amount for the crank 227b according to the driving data. The crank 227b drives the electrode assembly 11 to move toward the inside of the bracelet according to a rotation amount, so as to produce an effect of pressing the skin surface of the human body tightly.

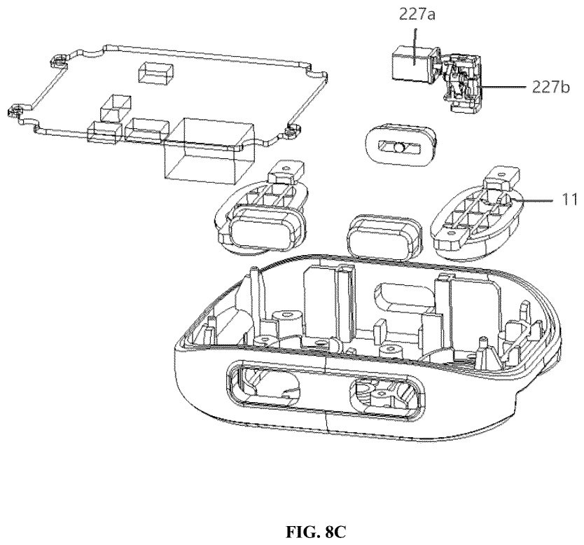

As shown in FIG. 8A and FIG. 8B, the motor 227a drives the crank 227b to rotate, and the crank 227b drives the electrode assembly 11 to move to produce an extrusion effect.

As shown in FIG. 8C, FIG. 8C is an expanded structural diagram of the driving device 22 of the foregoing seventh manner according to an example of an embodiment of the present disclosure. The structural diagram is only in an example form. In actual application, the structural diagram may be in another form, which is not specifically limited herein.

In an eighth manner, the driving device 22 includes a motor 228a, a gear 228b that is connected to the motor 228 an and that has a screw 228c, a screw nut 228d that is movably disposed on the screw 228c, and a slider 228e that is disposed on the screw nut 228d. The gear 228b drives the screw 228c to rotate when the motor 228a is driven, the screw nut 228d drives the slider 228e to move when the screw 228c is rotated, so as to drive the electrode assembly 11 to press the skin surface, and the motor 228a is electrically connected to the controller 00.

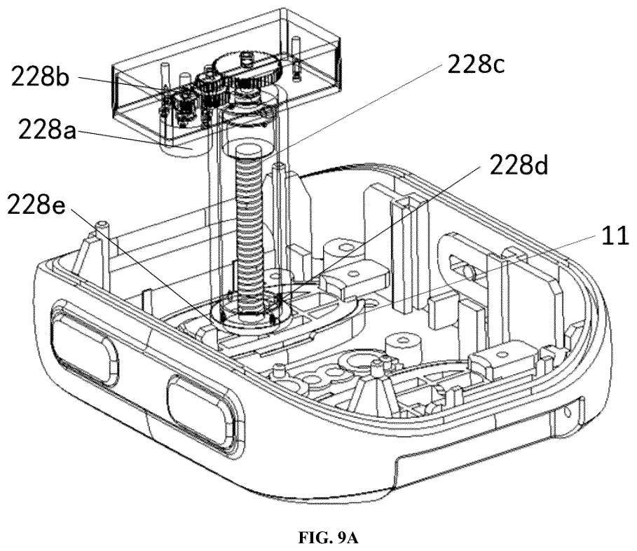

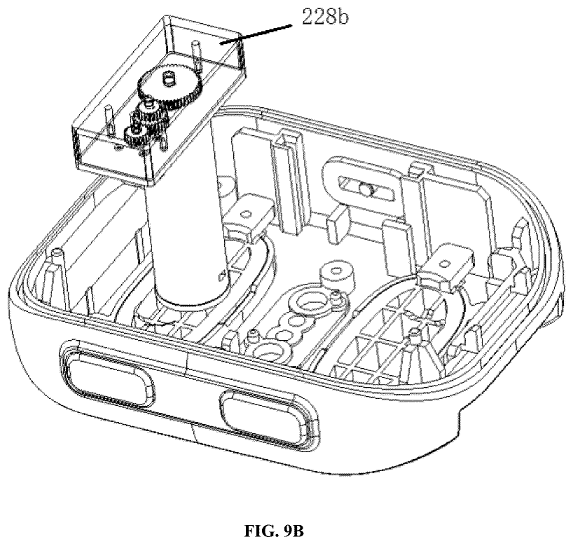

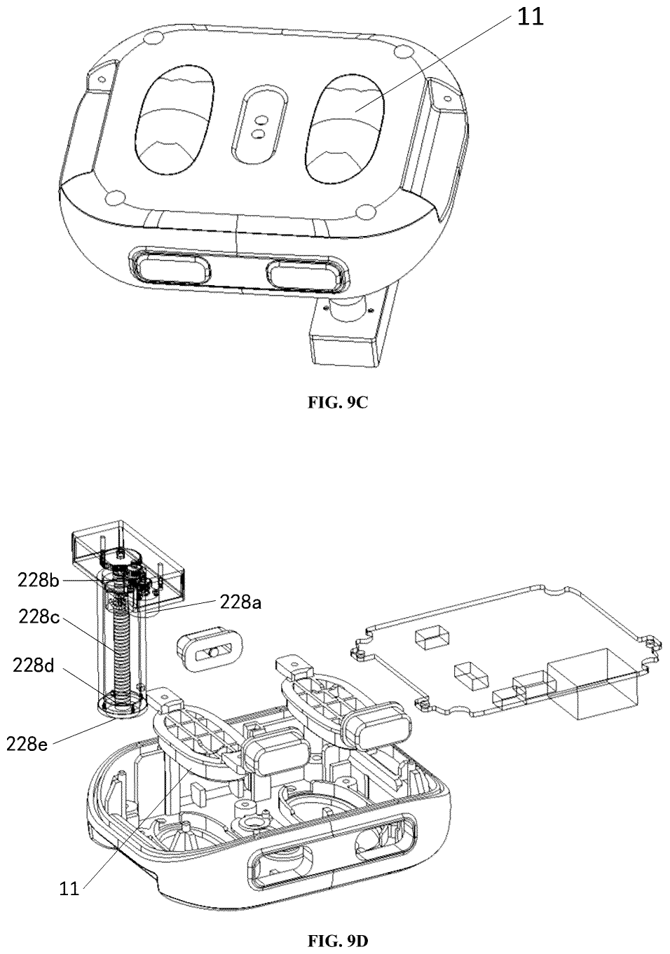

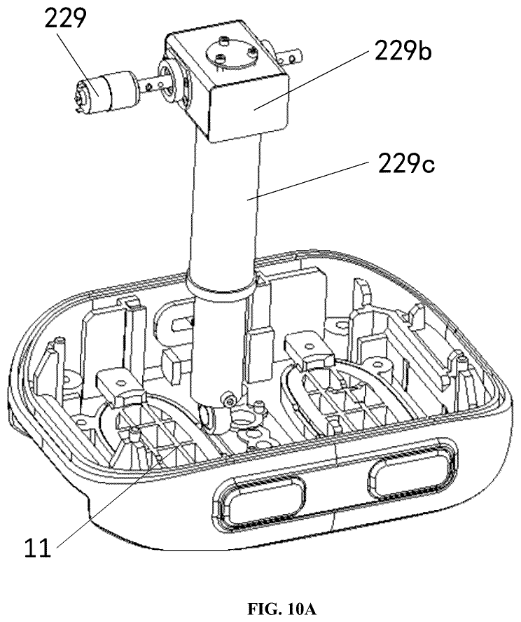

As shown in FIG. 9A and FIG. 9B, the motor 228a determines a rotation amount for the gear 228b according to the driving data. The screw 228c drives the screw nut 228d to generate an upper and lower displacement, so as to drive the electrode assembly 11 toward the inner side of the bracelet by using the slider 228e, to produce an effect of pressing the skin surface of the human body. Specifically, there are multiple gears 228b, and a deceleration effect is implemented by using a function of multiple gears.

As shown in FIG. 9D, FIG. 9D is an expanded structural diagram of the driving device 22 of the foregoing eighth manner according to an example of an embodiment of the present disclosure. The structural diagram is only in an example form. In actual application, the structural diagram may be in another form, which is not specifically limited herein.

In a ninth manner, the driving device 22 includes a motor 229, a worm 229b connected to the motor 229, and a screw rod 229c connected to the worm 229b. The screw rod 229c drives the electrode assembly 11 to press the skin surface by using a screw nut on the screw rod 229c when the worm 229b rotates, and the motor 229 is electrically connected to the controller 00.

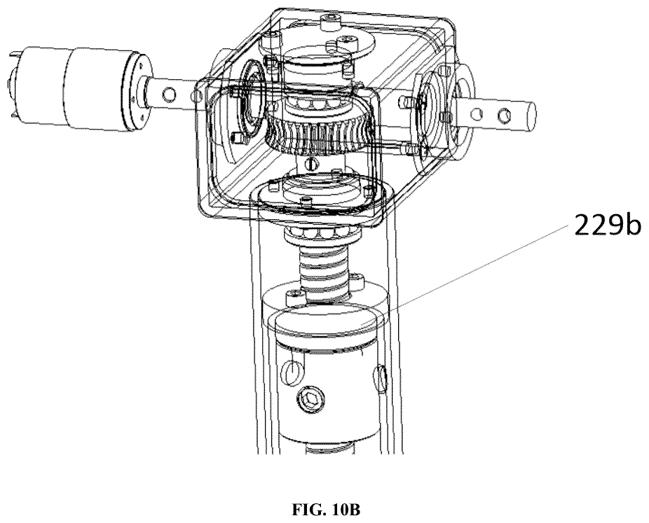

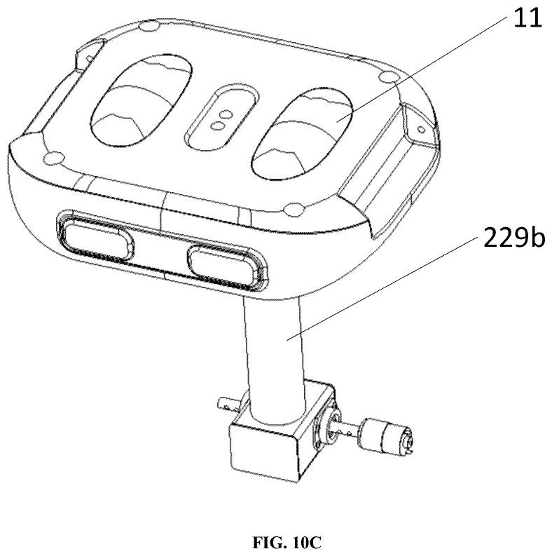

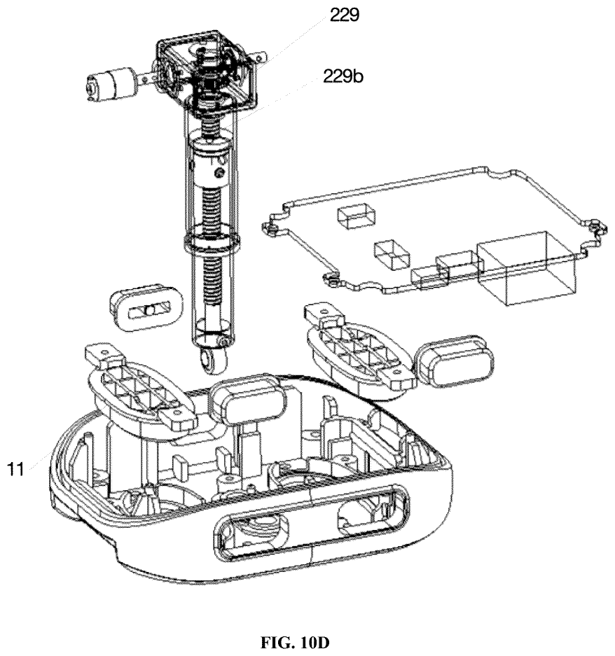

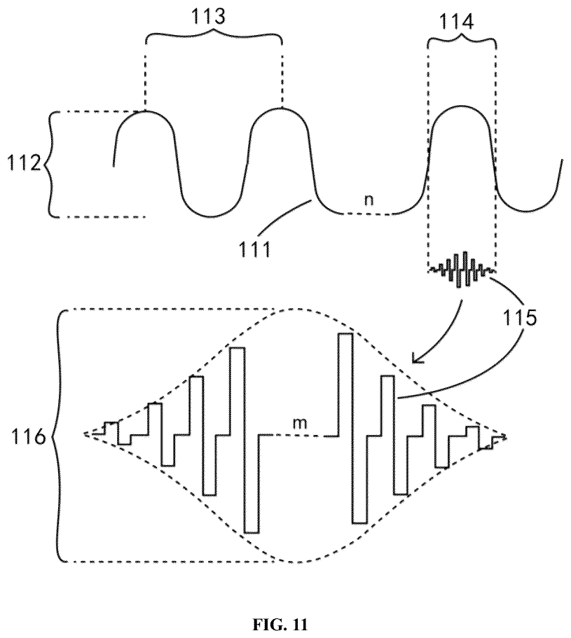

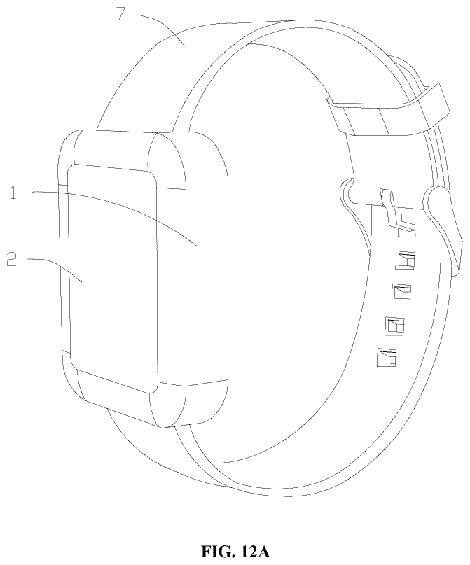

As shown in FIG. 10A and FIG. 10B, the motor 229 determines a rotation amount for the worm 229b according to the driving data. The worm 229b drives the turbine to rotate according to a rotation amount, so that when the turbine and the screw rod 229c rotate coaxially, the screw rod 229c drives the electrode assembly 11 to press the skin surface by using the screw nut on the worm 229b to rotate.

As shown in FIG. 10D, FIG. 10D is an expanded structural diagram of the driving device 22 of the foregoing ninth manner that cooperates with the ninth manner driving device according to an embodiment of the present disclosure. The structural diagram is only in an example form. In actual application, the structural diagram may be in another form, which is not specifically limited herein.

Regardless of the foregoing implementation manners, the bracelet provided in this embodiment of the present disclosure can implement a better electrical stimulation control effect by cooperating with an air pressure sensor 20.

In actual application, the air pressure sensor 20 may detect an air pressure value generated by the driving device 22, and transmit the air pressure value to the controller 00. In this way, the controller 00 may determine, according to the air pressure value and the current contact pressure value of the electrode assembly 11, the driving data for the driving device 22 and an electrical stimulation signal for implementing electrical stimulation for the electrode assembly 11.

Using the first airbag 221b and the air pump 221a shown in FIG. 2B and FIG. 2C as an example as the driving device 22, the air pressure sensor 20 may be disposed in the first airbag 221b. In this way, the air pressure sensor 20 can detect an internal air pressure value of the first airbag 221b in real time. Then, the controller 00 may determine, according to the air pressure value and the contact pressure value measured by the pressure sensor 30, the driving data adapted to the air pump 221a.

In this embodiment of the present disclosure, the controller 00 reads the measured value of the air pressure sensor 20 in the first airbag 221b, so as to effectively control an air pressure in the first airbag 221b by using the air pump 221a. On the one hand, an accurate instantaneous pressure fluctuation curve may be used to simulate a physical point function of the Chinese medicine, and excitement of the median nerve is enhanced by the physical point function, thereby significantly improving effectiveness of stimulation. On the other hand, the air pressure change in the first airbag 221b can be quickly switched, the hardness of the first airbag 221b and a skin depth change of a top concave skin can be operated, and a physical repeatedly pressing and vibration rubbing function can be simulated. The function cooperates with the electrical stimulation, and can effectively activate a median nerve in a deep part of the wrist of the human body, so as to achieve a superposition effect of an auxiliary massage, an acupuncture, or an electric moxibustion in traditional Chinese medicine diagnosis and treatment. Therefore, a practicability of the bracelet is further improved.

It should be noted that the air pump 221a herein may further adjust, according to different electrical stimulation modes, an air pressure in the first airbag 221b according to a curve. For example, the air pump 221a implements a continuously stable pressure control of the first airbag 221b.

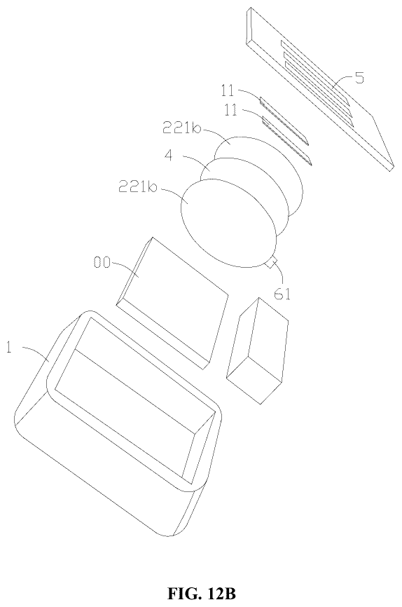

Herein, the electrical stimulation is implemented with reference to a cooperation between the controller 00, the air pump 221a, the first airbag 221b, the pressure sensor 20, and the pressure sensor 30. In a process in which the first airbag 221b is driven by the air pump 221a, the pressure sensor 30 may record a skin pressure at the electrode assembly 11 currently caused by the wrist strap 7 (which is referred to herein by a wrist strap pressure). The controller 00 calculates, based on the wrist strap pressure, a sine-like curve of an optimal pressure applied to the first airbag 221b. As shown in FIG. 11, in a case in which a relatively large pressure change indicated by a curve peak changes, an electrical stimulation is applied to the electrode assembly 11, thereby further improving an effectiveness of the electrical stimulation. This mainly considers that a relatively large pressure change enables a bracelet to press the skin surface of the human body tightly. In this way, in a case of a same energy stimulation, the stimulation effect of the bracelet may be better.

Specifically, a curve 111 shown in FIG. 11 represents a pressure change curve in the first airbag 221b. 112 represents an amplitude pressure curve between a wave peak and a wave valley of the first airbag 221b, 113 represents a duration of one cycle of filling and deflation of the first airbag 221b, 114 represents an electrical stimulation operating period when the air pressure of the first airbag 221b reaches a half of the amplitude, 115 represents an electrical stimulation waveform curve, and 116 represents a maximum amplitude difference of the electrical stimulation waveform curve.

In addition, an outline of a dotted line in the curve 115 represents an envelope range of an concussion curve of 115 that varies over time. As described herein, an amplitude difference of the electrical stimulation increases to a top as the pressure rises, and the amplitude of the electrical stimulation decreases as the pressure decreases.

As can be learned from FIG. 11, a detail waveform of an electrical stimulation herein is a two-way square wave. For example, the two-way square wave is released by using an electrode plate fixed on the first airbag 221b. This two-way square wave can ensure that an integral area under a curve is 0. Because an area covered by a square wave that is positive or negative is offset from each other, there is no charge accumulation, thereby further improving an electrical stimulation physiotherapy effect. For example, when an action is performed on a Neiguan acupuncture, further improving a treatment effect of preventing dizziness and preventing emesis. In addition, a sine-like curve adjustment manner used herein may further cause a user to pay attention to a respiration frequency, and discomfort caused by a car may be relieved to a certain extent by shifting attention.

In addition, FIG. 11 is only a specific example in which m and n are respectively used to indicate a quantity of repetition times of a pulse signal and a quantity of repetition times of a pressure signal, and values of m and n are not specifically limited herein.

In an actual application, to further improve use experience of different users, the bracelet provided in this embodiment of the present disclosure may prompt the user to adjust when contact strength is excessively large or excessively small, that is, the wrist strap 7 may be an adjustable and loose belt. This is mainly because a low pressure causes insufficient contact force between the electrode and the skin surface, which leads to a large contact resistance of the electrode, a decrease in the effectiveness of electrical stimulation, and an increase in skin fever. A high pressure causes discomfort to a patient. To solve the foregoing problem, the controller 00 herein may analyze an pressure data recorded by the pressure sensor 30 and adjust pressing states of the bracelet.

Herein, when the controller 00 determines that a contact pressure value between the electrode assembly 11 and the skin surface of the human body is less than a preset pressure value, the controller 00 may generate a reminder signal that is used to remind the user to adjust the bracelet, and prompt the user to adjust the wearing by using a display screen 33, as shown in FIG. 1.

In actual use of the bracelet, the bracelet further includes a protrusion element disposed on the first airbag 4 configured to press the acupuncture point. The protrusion element can be a protrusion or a metal piece, etc., which achieve massage during the inflation of the first airbag 4. In actual use of the bracelet, the bracelet further includes a second airbag 4 configured to press the acupuncture point, that is, after the electric stimulation is implemented by using the first airbag 221b to drive the electrode assembly 11 to move, the user may further massage the stimulated acupuncture point by using the second airbag 4, so as to further stimulate the acupuncture point to achieve a better stimulation effect. In other embodiment, the protrusion element can be disposed on the second airbag 221b, the wrist strap 7, or the dial 1, and a position of the protrusion element is not specifically limited herein.

The second airbag 4 may not be connected to the air pump 221a, and the user may press the second airbag 4 to stimulate the acupuncture point according to a requirement. Certainly, the second airbag 4 may further be connected to the air pump 221a, so as to implement an inflation under an action of the air pump 221a for use by the user. In addition, the second airbag 4 may be consistent with a working mode of the first airbag 221b, that is, a synchronization action with the first airbag 221b is implemented after the first airbag 221b is inflated and deflated. The first airbag 221b and the second airbag 4 may be separately inflated and deflated, so as to provide the user with more usage modes and improve usage experience.

In actual application, there may be multiple first airbags 221b, and a plurality of electrode assembly 11, the number of the first airbags 221b is equivalent to the number of the electrode assembly 11 b. An electrode assembly 11 is correspondingly configured with a first airbag 221b, and a size of the first airbag 221b may be rounded larger than a size of a corresponding one electrode assembly 11. For example, the first airbag 221b may extend in a direction of the wrist strap 7, that is, may occupy an inner side of the entire wrist strap 7. Certainly, in another embodiment, the number of the first airbags 221b may be one. In this case, a length and a width of the electrode assembly 11 may be relatively increased, so as to effectively press the acupuncture point on the wrist. Specifically, there may be two first airbags 221b, and are respectively located on two sides of the second airbag 4.

In an embodiment, both the first airbag 221b and the second airbag 4 are inflatable and deflatable airbags, that is, the first airbag 221b and the second airbag 4 may perform a pressing stimulation and massage on an acupuncture point on the wrist of the user by inflating and deflation.

Specifically, in an embodiment, both the first airbag 221b and the second airbag 4 are provided with an inflation and deflation opening 61. In another embodiment, the first airbag 221b and the second airbag 4 may be in an integral structure, and the first airbag 221b and the second airbag 4 are connected to each other. In this way, the inflation and deflation opening 61 is disposed on the first airbag 221b and/or the second airbag 4, and the inflation and deflation opening 61 is connected to the air pump 221a to implement charging and discharging of the first airbag 221b and the second airbag 4.

The display screen 33 in this embodiment of the present disclosure may further implement, based on application requirements of different users, specific display of various data such as an intensity of the electrical stimulation, a squeezing strength of the driving device 22, an countdown protection information, a breathing mode, or user health data. It can be understood, through the intensity of the electrical stimulation, the squeezing strength of the driving device 22, and the countdown protection information, a long time of the pressure provided to the skin surface or a long time of the electrical stimulation provided to the skin surface can be avoid. It can be understood, the squeezing strength of the driving device 22 raises, a pressing intensity of the electrode assembly 11 on the skin surface can raise too.

In addition, the display screen 33 in this embodiment of the present disclosure may further be a touch display screen 2, and a control result of the target task in response to a touch control instruction of the user for the target task: and the control result includes one or more of changing the intensity of the electrical stimulation, the squeezing strength of the driving device 22, the countdown protection information, and time under the touch control instruction. This is not specifically limited in the present disclosure.

It should be noted that, in this embodiment of the present disclosure, the display screen 33 with a simple function and the touch display screen 2 may be disposed simultaneously, or the display screen 33 with a touch function may be disposed. In this way, when a touch operation needs to be performed, a touch function may be implemented by an unlocking operation provided on the touch display screen, or a small touch partition may be disposed on the display screen 33 to support the touch function, which sets no specific limitation thereto.

Considering that an excessively high or excessively low temperature may cause user discomfort in an actual electrical stimulation process, a temperature measurement needs to be performed herein with reference to a temperature sensor 40 to set a safe temperature wall for the electrical stimulation. The temperature sensor 40 in the wrist strap provided in this embodiment of the present disclosure is electrically connected to the controller 00, and may detect a temperature value between the electrode assembly 11 and the skin surface of the human body, so that the controller 00 determines an electrical stimulation policy for the electrode assembly 11 according to the temperature value, and/or determines a temperature adjustment policy for the driving device 22.

For example, the temperature sensor 40 may be disposed near the electrode assembly 11 to determine a temperature value of the electrode assembly 11. Herein, if the temperature measured by the temperature sensor 40 reaches an improper line (for example, 39 degrees), in this case, the controller 00 may change a stimulation mode to a comfort mode, set the electrical stimulation intensity to an intermediate level, send a sound and a vibration prompt based on a reminder component, and may also prompt, by using the display screen 33, a change of the electrical stimulation policy. If the temperature reaches the guard line (for example, 41 degrees), in this case, the controller 00 may change the stimulation mode to the comfort mode, and the intensity of the electrical stimulation is set to a minimum value. At the same time, the controller 00 gives a sound and a vibration prompt based on the reminder component, and may further prompt the user by using the display screen 33 to show a change of the current temperature and the electrical stimulation policy.

It can be learned that, when determining that the temperature value exceeds a preset temperature threshold, the controller 00 in this embodiment of the present disclosure may replace the intensity of the electrical stimulation against the electrode assembly 11, for example, reduce the intensity of the electrical stimulation. At the same time, the controller 00 may remind the user in time by a reminder component.

In actual application, as shown in FIG. 1, the bracelet provided in this embodiment of the present disclosure may further be provided with a heating component 44 (for example, a heater). The heating component 44 is electrically connected to the controller 00. In this way, when the controller 00 determines that heating needs to be performed on the driving device 22, the heating component 44 is controlled to perform heating on the driving device 22.

For describing a specific heating principle easily, an example of the air pump 221a cooperating with the first airbag 221b may be described below.

With respect to the heating of the first airbag 221b, the heating is mainly used to simulate a hot moxibustion, and further excites a median nerve by increasing the temperature, so as to improve an electrical stimulation effect. In addition, sweating is induced by a temperature change at the skin surface contact on a top of the airbag, and sweat liquid has conductivity, so that a dry electrode surface of the electrode assembly 11 is wet by using a salt solution of the sweat liquid, and a contact resistance of the electrode assembly 11 is reduced, thereby significantly improving the stimulation effectiveness.

In actual application, the controller 00 in this embodiment of the present disclosure may obtain, by reading a temperature value of the temperature sensor 40, a temperature change situation in the first airbag 221b, and control a heater based on the temperature change situation, so that the temperature in the first airbag 221b is stable or fluctuates according to a curve, thereby achieving a stable and continuously effective electrical stimulation.

In an actual application, a heating situation may be worse with a wearing time of the bracelet raising. In this case, the bracelet may be cooled by using a refrigeration component 55, as shown in FIG. 1.

It should be noted that, in this embodiment of the present disclosure, a technical purpose of applying an electrical stimulation signal to the electrode assembly 11 may be implemented based on a determining result of a preset electrical stimulation condition. The preset electrical stimulation condition herein may be considered based on a single factor. For example, the electrical stimulation may be applied only when it is determined that the contact pressure is large enough, or may be determined based on multiple factors, for example, the electrical stimulation is applied only when it is determined that a contact pressure is large enough and a sensing temperature is not high enough. Specifically, the electrical stimulation condition may be comprehensively determined with reference to user health data, atmospheric pressure change data, or the like, which is not specifically limited herein.

As shown in FIG. 1, in one aspect, the bracelet provided in this embodiment of the present disclosure may implement a switching on/off control for the bracelet based on a switch component 66, or implement a switching on/off for an electrical stimulation mode, intensity, and the like. On the other hand, relative intensity adjustment may be implemented based on a rotary encoder 77. The following two aspects are described in detail.

According to a first aspect, when the switching on/off control is performed based on the switch component 66, the switch component 66 herein may acquire a switch signal triggered by a user, and determine, by means of analysis processing by the controller 00, a switch state for the bracelet or an electrical stimulation mode for the electrode assembly 11.

In actual application, the switch component 66 may control, by using a response of a first switch, a switch state of the bracelet or a switch of an electrical stimulation mode of the electrode assembly 11, or may control, by using a response of a second switch, an electrical the intensity of the electrical stimulation of the electrode assembly 11.

It should be noted that the foregoing switch setting is only a specific example. In a specific application, for example, a singal switch may be separately disposed to implement an electrical stimulation mode, or the electrical stimulation mode and the electrical stimulation intensity may be integrated into one switch operation, which is not specifically limited herein.

Regardless of whether the first switch, the second switch, or another singal switch may be disposed on a side of the dial, for example, a plurality of openings disposed on the side of the dial are used to insert the plurality of switches, and an external shape of each switch may be, for example, a circle, an oval, or a square, which is not specifically limited herein. The switch may be an entity button switch, or may be a virtual touch switch, which is not specifically limited herein.

According to a second aspect, when the switching on/off control is performed based on the rotation encoder 77, the rotation encoder 77 determines an intensity of the electrical stimulation of the electrode assembly 11 based on a variation indicated by a rotation adjustment operation in response to the rotation adjustment operation, so that the controller 00 applies an electrical stimulation signal matching the intensity of the electrical stimulation to the electrode assembly 11. Alternatively, the squeezing strength of the driving device 22 is determined, the pressing intensity of the electrode assembly 11 is determined too, so that the controller 00 applies a matching pressing force to the electrode assembly 11. Alternatively, a time at which the electrode assembly 11 exits is determined or a countdown protection information is determined, so that the controller 00 applies a matching pressing time to the electrode assembly 11.

It can be learned that a corresponding rotation control operation is implemented based on the rotation encoder 77, which further improves practicality in different application scenarios.

To further improve practicality of the bracelet provided in this embodiment of the present disclosure, a breathing adjustment component 88 may be disposed herein. As shown in FIG. 1, the breathing adjustment component 88 is electrically connected to the controller 00. In this way, in a case in which the electrical stimulation is applied to the skin surface of the human body, the breathing adjustment component 88 may remind the wearing user to adjust respiration according to a preset breathing mode, so as to alleviate a discomfort such as when the user is in a seasick state.

A health data collection component 99 may be further disposed in the bracelet herein. As shown in FIG. 1, the health data collection component 99 is electrically connected to the controller 00. The health data collection component 99 may collect, in real time or periodically, health data of the user.

The health data includes at least one or more of the following data: a temperature value, a blood pressure value, a blood oxygen value, and a heartbeat value.

Based on the foregoing health data collection, the controller 00 may further provide a health analysis result and/or a health report according to the health data. In addition, in this embodiment of the present disclosure, a support may be provided in a timely manner when a user is in a slight improper condition. In addition, when the user is in a obvious improper condition, an emergency rescue method (such as reporting a dangerous signal to an emergency contact terminal bound to a bracelet) may be used to help the user survive in a difficult condition.

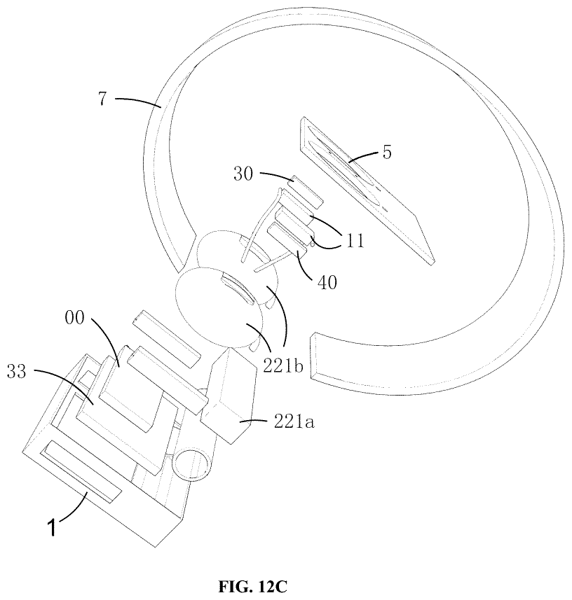

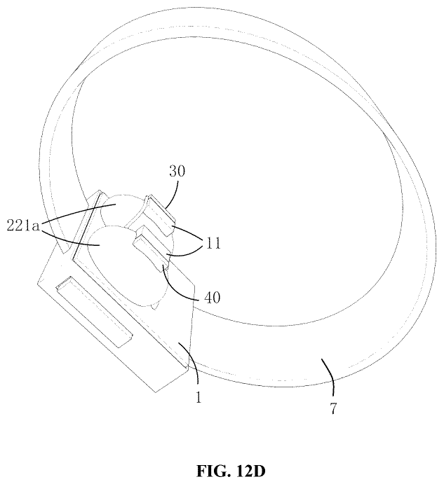

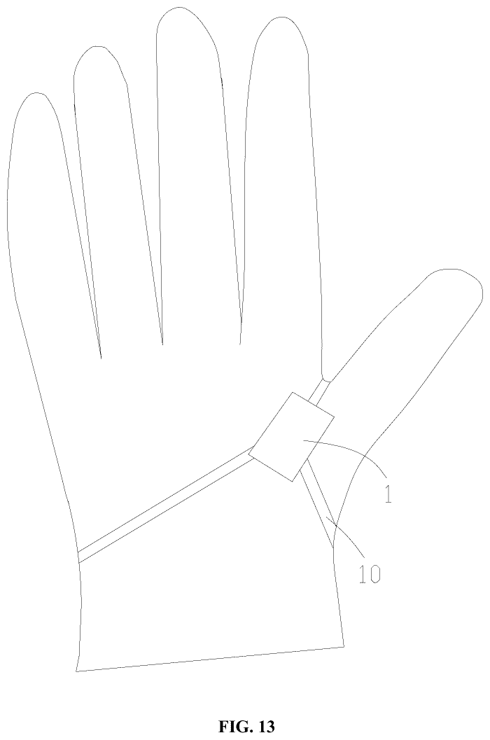

In the foregoing embodiments of the present disclosure, no specific identifier is provided for a base part such as a bracelet. To further understand the embodiments of the present disclosure, a specific structure of the first airbag 221b driving the electrode assembly 11 is described with reference to a schematic structural diagram shown in FIGS. 12A˜12D in the following with reference to an example.

The bracelet in this embodiment of the present disclosure further includes a housing 1, and the housing 1 is disposed on the wrist strap 7. The controller 00 is a circuit board, and is disposed in the housing 1. The touch display screen 2 is disposed on the housing 1, and the first airbag 221b, the second airbag 4, the air pump 221a, the switch assembly 66, the air pump, the airbag, the temperature sensor 40, the pressure sensor 30, and the like may be disposed in the housing 1. The wrist strap 7 may be a loosely or elastic belt by means of mechanical structure adjustment, for example, may be a slightly elastic and adjustable nylon knitted belt, and has excellent features such as skin-friendly property, low sensitivity, and breathability.

In actual application, the driving device 22 is preferably disposed on the housing 1, so that the driving device 22 can be fixed and installed. Certainly, the driving device 22 may not be disposed on the controller 00, and is directly disposed on the wrist strap 7.

To facilitate information display, the touch display screen 2 is disposed on a top surface of the housing 1. The touch display screen 2 may timely remind the user to adjust and wear the bracelet. In addition, another related prompt such as low power may be performed.

To facilitate more direct electrical stimulation, a first opening 5 may be disposed on a bottom of the housing 1, the first airbag 221b and the second airbag 4 may protrude from the first opening 5 and outside the housing 1 when the first airbag 221b and the second airbag 4 are deformed, so as to drive the electrode assembly 11 to press the skin surface of the human body.

In an actual application, the first airbag 221b is bonded and integrated with the pressure sensor 30, the electrode assembly 11, and the temperature sensor 40. When the first airbag 221b is inflated, the pressure sensor 30, the electrode assembly 11, and the temperature sensor 40 project from a top of the first opening 5.

Considering that a material of a Polycarbonate (Polycarbonate, PC) has excellent characteristics such as high strength, elasticity coefficient, high impact strength, and a wide use temperature range, the housing 1 herein may be made of a PC material, and a second opening (not shown in the figure) disposed on the housing 1 and that is used for a touch display 2 and a switch assembly 66. Waterproof encapsulation is performed at the second opening and at a connection to another component by using encapsulation glue, for example, by using polyurethane glue. The housing 1 may further be made of a skin-friendly silicone rubber or a titanium alloy material, and is connected to another component by using a waterproof glue.

In actual application, the hand wearable apparatus may be further applied to the an area between a thumb and a forefinger (such as a Hegu point area), that is, a wearing part 10 is a wearing body, and the hand wearable apparatus is disposed on the wearing body. Preferably, the driving device 22 is disposed on the wearable body, and the electrode assembly 11 is disposed on the driving device 22, so as to directly drive, when the driving device 22 is driven, the electrode assembly 11 press the skin surface of the human body. In another embodiment, the electrode assembly 11 and the driving device 22 are disposed at approximately a relative position, or may drive the electrode assembly 11 to press the skin surface of the human body when the driving device 22 is driven.

In an embodiment of the wearable apparatus, the wearable apparatus may further include the controller 00 in an embodiment of the bracelet. The controller 00 may be fixed to the wearable body by using the housing 1, and the driving device 22 is disposed on the controller 00, which is the same as that in the bracelet.

Herein, when the user wears the wearable apparatus for the area between the thumb and the forefinger, that is, the electrode assembly 11 is close to the area between the thumb and the forefinger by using the driving device 22, and the Hegu point is a position that lets vitality of the human body pass and stay. By stimulating or massaging the area between the thumb and the forefinger, a function of calming and relieving pain, relaxing muscles and tendons, and an enhancing body resistance may be performed, and a good physical healing function is performed on the human body. In addition, by using the driving device 22, the electrode assembly 11 is squeezed into the skin surface of the human body, which can resolve a problem that electrode plates in an existing structure in which the Hegu point is electrically stimulated cannot be properly applied to the skin surface of the human body.

It should be noted that, in this embodiment of the present disclosure, all directional indications (such as up, down, left, right, front, and back). It is only used to explain a relative positional relationship, a motion condition, and the like between components in a specific posture (as shown in the accompanying drawings). If the specific posture changes, the directional indication changes accordingly.

In addition, descriptions in the present disclosure relating to “first”, “second”, and the like are used only for description purposes, and cannot be understood to indicate or imply relative importance or implicitly indicate a quantity of indicated technical features. Therefore, a feature defined with “first” and “second” may explicitly or implicitly include at least one feature. In addition, the “and/or” in this specification includes three solutions, using an and/or B as an example, including a technical solution A, a technical solution B, and a technical solution that both an and B meet. In addition, the technical solutions between the embodiments may be combined with each other. However, a person of ordinary skill in the art may be used as a basis for implementation. When a combination of the technical solutions is contradictory or cannot be implemented, it should be considered that a combination of the technical solutions does not exist, and is not within the protection scope required by the present disclosure.

The foregoing descriptions are merely preferred embodiments of the present disclosure, and are not intended to limit the patent scope of the present disclosure. Any equivalent structural transformation performed by using the content in the specification and accompanying drawings of the present disclosure or directly/indirectly applied in another related technical field is included in the protection scope of the present disclosure.

The above descriptions are only preferred embodiments of the present disclosure, and are not intended to limit the patent scope of the present disclosure. Any equivalent structural transformation made by using the content of the specification and the drawings of the present disclosure under the disclosure idea of the present disclosure, directly or indirectly applied to other related technical fields, shall all be included in the scope of patent protection of the present disclosure.

1. A hand wearable apparatus, comprising:
a wrist strap configured to be worn on a hand;
an electrode assembly disposed on the wrist strap, wherein the electrode assembly is configured to abut a skin surface of a human body and provide a micro current to the skin surface; and
a driving device disposed on the wrist strap, wherein the driving device is configured to drive the electrode assembly to press against the skin surface for providing the micro current and massage the skin surface of the human body simultaneously.

2. The hand wearable apparatus according to claim 1, wherein the driving device comprises a first airbag and an air pump configured to inflate or deflate the first airbag, and the first airbag is configured to drive the electrode assembly to press against the skin surface during inflation or deflation.

3. The hand wearable apparatus according to claim 1, wherein the driving device comprises an air pump, an air pipe and a cylinder connected to the air pump by using the air pipe, and the cylinder is configured to drive the electrode assembly to press against and massage the skin surface when the air pump blows air to the cylinder.

4. The hand wearable apparatus according to claim 1, wherein the driving device comprises a motor, a screw connected to an output shaft of the motor, a screw nut cooperation with the screw, and a slider disposed on the screw nut, and the slider is configured to drive the electrode assembly to press against the skin surface when the screw rotates.

5. The hand wearable apparatus according to claim 1, wherein the driving device comprises a motor, a gear connected to the motor, and a rack engaged with the gear; and the rack is configured to drive the electrode assembly to press against the skin surface when the gear rotates.

6. The hand wearable apparatus according to claim 1, wherein the driving device comprises an electric push rod, and when the electric push rod [works] actuates, the electrode assembly is driven to press against the skin surface.

7. The hand wearable apparatus according to claim 1, wherein the driving device comprises a coil and a magnet, and when the coil is powered on, the magnet moves to drive the electrode assembly to press against the skin surface.

8. The hand wearable apparatus according to claim 1, wherein the driving device comprises a motor and a crank connected to the motor, and the crank is configured to drive the electrode assembly to press against and message the skin surface when the motor is driven.

9. The hand wearable apparatus according to claim 1, wherein the driving device comprises a motor, a gear connected to the motor, a screw, a screw nut movably disposed on the screw, and a slider disposed on the screw nut;
wherein the gear is configured to drive the screw to rotate when the motor is driven, and the screw nut is configured to drive the slider to move when the screw is rotated, so as to drive the electrode assembly to press against the skin surface.

10. The hand wearable apparatus according to claim 1, wherein the driving device comprises a motor, a worm connected to the motor, and a screw rod connected to the worm; and the screw rod drives the electrode assembly to press against the skin surface when the worm rotates.

11. A bracelet, comprising:
a wrist strap;
a dial connected to the wrist strap;
an electrode assembly, wherein the electrode assembly is configured to press a skin surface of a human body and provide a micro current to the skin surface;
a driving device, wherein the driving device is configured to drive the electrode assembly to press against the skin surface for providing the micro current and massage the skin surface simultaneously; and
a controller electrically connected to the driving device and the electrode assembly respectively.

12. The bracelet according to claim 11, wherein the electrode assembly is disposed on a driving device, and the driving device is disposed on the wrist strap or the dial.

13. The bracelet according to claim 11, wherein the electrode assembly is disposed opposite to a driving device, and the driving device is disposed on the wrist strap or the dial.

14. The bracelet according to claim 11, wherein the driving device comprises at least one first airbag and at least one air pump electrically connected to the controller, and the at least one first airbag is configured to drive the electrode assembly to press against and message the skin surface when the at least one air pump is driven, wherein the electrode assembly is located at a position corresponding to an area adjacent to a neriguan acupuncture point of the human body.

15. The bracelet according to claim 14, wherein the at least one first airbag and the electrode assembly are disposed in the wrist strap or the dial.

16. The bracelet according to claim 14, wherein the at least one first airbag is disposed in the dial, and the electrode assembly is disposed on the wrist strap.

17. The bracelet according to claim 14, wherein the at least one first airbag is disposed on the wrist strap, and the electrode assembly is disposed on the dial.

18. The bracelet according to claim 14, wherein the at least one first airbag comprises two first airbags, each first airbag is disposed on both the wrist strap and the dial, and the electrode assembly is disposed on the wrist strap or the dial.

19. The bracelet according to claim 14, wherein the bracelet further comprises a second airbag configured to press an acupuncture point and/or a protrusion element for pressing and messaging an acupuncture point simultaneously.

20. The bracelet according to claim 19, wherein the second airbag is an inflatable airbag.

21. The bracelet according to claim 19, wherein the first airbag and the second airbag are in an integral structure, the first airbag communicates with the second airbag, and an inflation and deflation opening is disposed on the first airbag and/or the second airbag.

22. The bracelet according to claim 14, further comprising a pressure sensor and an air pressure sensor, wherein the pressure sensor and the air pressure sensor are electrically connected to the controller; the pressure sensor is configured to obtain a contact pressure value between the electrode assembly and the skin surface and transmit the contact pressure value to the controller;
the air pressure sensor is configured to generate a barometric pressure value by detecting the driving device and transmit the barometric pressure value to the controller; and
the controller is also configured to provide a driving data for the driving device and an electrical stimulation signal for the electrode assembly according to the barometric pressure value and the contact pressure value, such that the electrode assembly press against the skin surface for providing the micro current and massage the skin surface simultaneously.

23. The bracelet according to claim 22, wherein the controller is further configured to determine the contact pressure value between the electrode assembly and the skin surface of the human body is less than or greater than a preset pressure value, and generate a reminder signal for reminding a user to adjust the bracelet when the contact pressure value between the electrode assembly and the skin surface is less than or greater than the preset pressure value.

24. The bracelet according to claim 11, further comprising a display screen electrically connected to the controller, wherein the display screen is configured to display a result of a target task in response to displaying an instruction of a user to complete the target task, and the display result comprises at least one or more of the following information: an intensity of the micro current, a squeezing strength of the driving device, a breathing mode, or user health data.

25. The bracelet according to claim 24, wherein the display screen is a touch display screen; and the touch display screen is configured to display a control result of the target task in response to displaying an instruction to the user to complete the target task; and the control result comprises one or more of changing the intensity of the micro current, and the squeezing strength of the driving device.

26. The bracelet according to claim 14, wherein a pressure sensor electrically connected to the controller is disposed in the first airbag; the pressure sensor is configured to detect a pressure value inside the first airbag and transmit the pressure value to the controller, and the controller determines air pressure controlling data for controlling the air pump according to the pressure value and the contact pressure value.

27. (canceled)

28. (canceled)

29. The bracelet according to claim 11, further comprising a health data collection component electrically connected to the controller; and the health data collection component configured to collect, in real time or periodically, health data of a user, wherein the health data comprises at least one or more of the following data; a temperature value, a blood pressure value, a blood oxygen value, and a heartbeat value; and the controller provides a health analysis result and/or a health report based on the health data.

30. The bracelet according to claim 11, wherein the controller is also configured to acquire driving data for the driving device; the controller is also configured to apply an electrical stimulation signal to the skin surface via the electrode assembly when the driving device drives the electrode assembly to press against the skin surface; and
the controller is also configured to apply the electrical stimulation signal to the electrode assembly when a preset electrical stimulation condition is reached.

31. (canceled)

32. The bracelet according to claim 14, further comprising a temperature sensor, a heating component, and a refrigeration component electrically connected to the controller; wherein the temperature sensor is configured to detect a temperature value between the electrode assembly and the skin surface, and transmit the temperature value to the controller; the controller is configured to determine a temperature adjustment policy for the at least one first airbag according to the temperature value, and/or an electrical stimulation policy for the electrode assembly; and
the controller is further configured to control the heating component to heat air in the at least one first airbag; and the refrigeration component is configured to cool the bracelet when an electrical stimulation is applied to the skin surface by the electrode assembly.

33. A wearable apparatus for an area between a thumb and a forefinger, comprising:
a wearing belt used for wearing;
an electrode assembly disposed on the wearing belt, wherein the electrode assembly is configured to abut a skin surface of a human body and provide a micro current to the skin surface, and the electrode assembly is located at a position corresponding to the area between the thumb and the forefinger when the wearing belt is worn on a hand of a user, the area is Hegu point area; and
a driving device disposed on the wearing belt, wherein the driving device is configured to drive the electrode assembly to press against the skin surface for providing the micro current and massage the skin surface simultaneously.
